(19) United States
(12) Patent Application Publication
(10) Pub. No.: US 2023/0211075 A1
(43) Pub. Date: Jul. 6, 2023

(54) PATCH INJECTION DEVICE WITH RELEASE LINER REMOVAL

(71) Applicant: Ypsomed AG, Burgdorf (CH)

(72) Inventors: Marc Fiechter, Konolfingen (CH); Serge Bart, Bern (CH); Stefan Burren, Schwarzenburg (CH); Regina Marbert, Rutschelen (CH); Balz Schaerer, Oberwil bei Buren (CH)

(21) Appl. No.: 18/175,930

(22) Filed: Feb. 28, 2023

Related U.S. Application Data
(63) Continuation of application No. PCT/EP2021/072343, filed on Aug. 11, 2021.

(30) Foreign Application Priority Data
Sep. 9, 2020 (EP) 20195282.7

(51) Int. Cl. A61M 5/142

(52) U.S. Cl. CPC A61M 5/14248; A61M 2209/06

(57) ABSTRACT

A patch injection device comprising a housing, an adhesive substrate having a first surface with a first surface area for skin attachment and a second surface which is opposite the first surface attaching partly to the housing and a removable release liner. The surface area of the second surface attaching to the housing is smaller than the first surface area and the excess area defines a skirt of the substrate at least partially surrounding the housing. The first surface of the substrate attaches to the removable release liner which fully covers the first surface area of the substrate. Furthermore a pull tab is provided being part of the release liner and located in a cut-out of the skirt of the substrate such that a peel force is transmittable from the release liner to the housing at the location where the cut-out reaches the housing.

### CROSS-REFERENCE TO RELATED APPLICATIONS

This application claims priority to International Patent Application No. PCT/EP2021/072343, filed Aug. 11, 2021, entitled “PATCH INJECTION DEVICE WITH AN IMPROVED RELEASE LINER REMOVAL,” which in turn claims priority to European Patent Application No. 20195282.7, filed Sep. 9, 2020, entitled “PATCH INJECTION DEVICE WITH AN IMPROVED RELEASE LINER REMOVAL”, each of which is incorporated by reference herein, in the entirety and for all purposes.

### TECHNICAL FIELD

The current disclosure relates to a patch injection device including a housing with an adhesive substrate connected thereto and the adhesive substrate may be covered by a release liner. The adhesive substrate and the release liner may facilitate improved removal of the release liner from the patch injection device.

### BACKGROUND

Injection or infusion devices may be used for the subcutaneous delivery of fluid medicaments. Infusion devices such as infusion pumps may be used for medicament delivery according to a delivery profile including bolus injections superimposed on a basal rate for medicaments such as insulin. Injection devices such as injection pens may be used for single bolus type of injections according to a preset (fixed) dose or to an adjustable dose.

Patch injection devices may be provided with an adhesive patch such that they may be adhered to the skin of the patient. The patch injection device may be operated for the medicament delivery according to a delivery profile or for bolus type of injections. The dose may be delivered from a reservoir via a fluid path ending in a needle or cannula. The patch injection device may be attached to the skin of the patient using a substrate containing a skin adhesive. Prior to use, the skin adhesive may be covered by a release liner and the skin adhesive should allow for device removal from the skin after completion of the injection without tissue damage. Patch injectors may be configured for the delivery of large bolus volumes (for example large dose bolus injectors), including reservoirs with a volume above 1 mL, for example 5 mL or 10 mL. In such case the weight of the patch injector may call for an improved skin adhesion system.

The release liner may be removed by the user by holding the device in one hand and peeling the release liner with the other hand. Solutions where both hands are required, such as holding or peeling the substrate with one hand and holding or peeling the release liner with the other hand, may result in that the user cannot hold the device itself. This may lead to dropping the device while peeling the release liner thereby potentially damaging the device or wasting the drug. This may have an impact to the patients’ therapy and, moreover, an expensive medicament may be lost.

The substrate used for skin attachment may be a flexible substrate such that the substrate can accommodate to the contours of different types of patients and may be used at different anatomical locations on the body requiring a different shape of the injection device that a (rigid) substrate may not provide.

WO2017219154 discloses a patch injection device having a substrate for skin attachment and the substrates’ surface area equals the bottom surface area of the housing. The release liner extends beyond the substrate thereby forming a pull tab. The user may hold the device in one hand and grab the extended area of the release liner with his other hand for liner removal. The maximum size of the cartridge, defining to a large extent the weight of the device, may be restricted by the skin adhesive used combined with the limited substrate surface area available for skin contact.

WO2007122207 discloses a modular patch injection device with an adhesive substrate extending beyond the device housing. The release liner covers the adhesive and no pull tab is provided for removing the release liner. Liner removal may be cumbersome as both hands need to be used to grab both the substrate and the liner and no hand is free for holding the housing of the device.

WO2019008562 discloses an arrangement for a patch injection device using two separate liners and both hands are required for removing the two separate liners at once.

In EP2914314, the substrate including the skin adhesive forms a skirt surrounding the device. The substrate is flexible and includes a separate stiffener which may be stiff enough to resist a force on the substrate caused by peeling of the release liner.

### SUMMARY

Embodiments of the present disclosure provide a patch injection device with an improved device handling overcoming the disadvantages of the prior art by combining an increased contact surface for skin attachment with an improved release liner removal.

The patch injection device may include an adhesive substrate for skin attachment and the adhesive substrate defines a skirt surrounding the housing of the patch injection device. The substrate includes a cut-out whereas the release liner extends beyond the cut-out such that the release liner may be peeled by the user while holding the device. The cut-out extends to the housing of the device such that the peel off force is directly transmitted, or guided to the housing.

The current disclosure relates to a patch injection device including a housing and an adhesive substrate having a first surface with a first surface area for attachment to the skin and a second surface which is opposite the first surface attaching at least partially to the housing. The patch injection device includes a removable release liner. The surface area of the second surface attaching to the housing is smaller than the first surface area and the excess area may define a skirt for the substrate, which at least partially surrounds the housing. The first surface of the substrate attaches to the removable release liner which may fully cover the first surface area of the substrate. The patch injection device furthermore includes a pull tab that may be part of the release liner and which may be located in the cut-out of the skirt of the substrate such that a peel force is transmittable from the release liner to the housing at the location where the cut-out reaches the housing. In other words, the skirt of the substrate surrounding the housing may be interrupted at the location of the pull tab, and presents or includes the cut-out in the substrate with a width which may be at least equal to the width of a thumb of the patient.

The skirt at least partially surrounding the housing extends the area of the adhesive substrate beyond the area of the housing, and the excess area may improve the attachment to the skin such that a less strong skin adhesive may be used thus improving the desired removal of the device from the skin after use. The excess area may allow for the use of larger volume reservoirs or cartridges (for example 5 mL or 10 mL) to be used as the latter define a substantial part of the weight of the device and the excess area may avoid undesired detachment of the injection device before completion of the injection. As an alternative both an easy-to-remove skin adhesive and a large volume medicament reservoir may be used in combination with a skirt surrounding the housing.

The removable release liner may be provided as a sheet that at least fully covers the first surface area of the adhesive substrate and prevents undesired attachment of the patch injection device prior to use. The pull-tab may be provided, like the release liner, as a sheet and improves the removal of the release liner from the patch injection device. The pull-tab is part of the release liner, or at least connected to the release liner in a manner to transmit a release force applied to the pull tab to the release liner. The release liner may be reinforced by additional layers.

The location of the pull tab in the cut-out of the skirt may ensure that the pull tab locally extends beyond the skirt and can be easily grabbed by the user on both sides for liner removal. The cut-out in the skirt of the substrate may ensure that the pull tab is free standing and locally extending beyond the skirt or the housing of the device thus leaving both surfaces of the sheet forming the pull tab available for a user.

The peel force may be transmittable from the release liner to the housing where the cut-out reaches the housing. This may imply that the peel force is locally directly transmitted to the housing where the substrates’ cut-out is adjacent to the housing and the peel force may not be transmitted to the substrate or part of the substrate that is not adjacent to the housing. The latter part of the substrate that is not adjacent to the housing may be damaged, flexed or is compliant and the release of the release liner may require both hands of the user to peel the substrate from the release liner and/or peel the release liner from the substrate. The peel force may be directly transmitted to the housing, which is held by the user in one hand as the user grabs the pull tab with his other hand. The mechanical stress is locally concentrated at the interface between the release liner and the first surface of the substrate which may be locally supported or backed-up by a rigid housing. The concentration of the mechanical stress may form a starting point for controlled liner removal such that the release liner can be easily removed in one piece without tearing the liner and/or or the substrate. The user can hold the device in one hand and the pull-tab in the other hand. This in contrast to the prior art devices where two pull tabs, each connected to two parts of the release liner, must be held by the user using both hands and no hand may be free for holding the device which complicates handling, or may require more handling steps.

In another embodiment, the patch injection device may include two cut-outs or a plurality of cut-outs in the substrate that each reach to the housing. Two or more cut-outs in the substrate may provide that the device may be held in two different orientations for liner removal. As the device is held with one hand by the user, the two cut-outs may be positioned to facilitate liner removal either for left-handed or for right-handed users.

The release liner may partially extend beyond the adhesive substrate in the area of the cut-out, such that the surface area of the release liner is larger than the first surface area.

The release liner may follow the outer contour of the adhesive substrate except for the part where there is a cut-out in the substrate. The part of the release liner in the cut-out of the substrate may therefore provide a pull-tab. The release liner may follow the virtual contour of the substrate as if there was no cut-out. For example, the substrate may have a rectangular shape with one corner left out forming the cut-out. The release liner may have the full rectangular shape including all four corners and thus extends from the substrate in the area of the cut-out.

The patch injection may include an adhesive substrate which may be flexible. Flexible is defined as being non-rigid and capable to be compliant with a non-flat or curved surface. Flexible materials typically have a young’s modulus below 1 GPa, or below 0.2 GPa, or below 0.1 GPa. Examples are sheets of paper, or thin film materials constructed from a polyester such as PET, a polyamide or a polyolefenic material with a thickness below 0.3 mm.

A flexible substrate provides that the substrate may follow the contours of the patient’s body once adhered. In an embodiment, only the skirt surrounding the housing may be flexible and compliant with the shape of the patient’s body. The flexibility of the substrate ensures a good contact to the body and efficient use of a body adhesive thereby preventing release of the patch injection device before completion of the injection. In another embodiment, only the skirt area may include a skin adhesive and the area of the first surface opposite to the second surface area contacting the housing has no skin adhesive to avoid excessive perspiration during use. In another embodiment, two different types of skin adhesives or two different substrates may be used, one for the skirt and the other one for the center area.

The pull-tab may be integrally formed with the release liner or the pull-tab may be a separate component attached to the release liner.The pull-tab extends from the device, for example from the cut-out of the substrate for easy access to the user. The pull-tab may be a separate part attached to the release liner such that the pull-tab may extend even further from the device beyond the virtual contour of the substrate. The pull-tab may be constructed from a different material compared to the release liner with a higher tear strength, or the pull-tab may have a textured surface for giving a different haptic feeling to the user. Optionally, the pull-tab as a separate but attached component may have a different color or may be used for branding the product. A separate pull-tab may thus increase the versatility of the device. The pull-tab as a separate component may be sleeve shaped as the pull tab may be part of a needle cover sleeve.

Indicators such as arrows may be printed onto the pull-tab both for the integrally formed, and for the separate pull-tab.

The pull-tab may extend in a plane that is parallel to the first surface or parallel to the substrate. In another embodiment, the pull tab may extend in a plane that is angulated with respect to the first surface or may be perpendicular to the first surface.

The pull-tab for the patch injection device may include an opening or a hole forming a pull ring. The hole may be formed in the sheet of the release liner when the pull tab is integrally formed with the release liner, or the hole may be in the extension of the separate part forming the pull tab.

A hole in the pull tab, independent from being integrally formed with the release liner or being constructed as a separate part attached to the release liner, may provide that the tab can easily be gripped by the user to support liner removal. The hole may be circular or elliptical shaped, or have a different shape such as an arrow shape.

The peel force for the release liner of the patch injection device may be transmittable to the housing via the adhesive substrate at the location where the cut-out reaches the housing.

At the location where the cut-out reaches the housing, there is a border area where the housing and the substrate end, whereas the release liner continues to extend in the lateral direction or parallel to the bottom surface of the housing. Peeling the release liner by pulling the pull-tab may result in a direct load transfer from the release liner to the housing via the substrate. The flexible substrate will locally not flex or bend as the substrate is locally mechanically supported by a mechanically stiff housing or housing part. Thus by using the cut-out, flexing and deformation of the substrate during liner removal is avoided. Once the initial part of the release liner has been removed from the substrate adjacent to the cut-out, the further removal will be promoted as the peel-off force will be evenly distributed and the peel off starts from the defined starting point.

A cut-out that reaches the housing may be defined in that a part of the cut-out may reach (coincide with) the location where the second surface attaches to the housing or is adjacent to that location, either within the area of the skirt or within the second surface.

The cut-out for the skirt in the substrate may be arc-shaped.The cut-out of the substrate may have different shapes such as an arc-shape or an elliptical shape or a circular shape. The contour forming the shape is proximate or adjacent to the housing. The release liner extending from the cut-out may have indicators, for example an arrow, printed thereon for guiding the user to peel into a peel direction. Alternative to the arc shape, the cut-out may be semicircular shaped, S-shaped, C-shaped, or there is a linear edge for the cut-out. A linear cut-out may result in a triangular shaped cut-out.

The arc shaped cut-out of the skirt may be adjacent or locally congruent to the contacting surface between the housing and the substrate. Thus the edge of the surface area of the second surface attaching to the housing may be adjacent to the contour forming the cut-out.

The housing of the patch injection device includes at least one corner and the at least one corner is adj acent to the arc shaped cut-out of the skirt. The corner of the housing may be a rounded corner and located in a plane parallel to the substrate. In another embodiment the corner may be located in a plane perpendicular to the substrate. In yet another embodiment, the corner is rounded in both planes forming a cup shaped corner.The release liner contacting the substrate may be split into two separate components each having a separate pull tab.

The adhesive substrate may be partially stiffened in the skirt surrounding the housing. To further improve the removal of the release liner in combination with a flexible substrate, a mechanical support may be provided or added to the substrate. The mechanical support may be provided locally on the part of the substrate that is adjacent to the cut-out. A variable stiffness substrate may be achieved by locally applying stiffeners. Those stiffeners may be formed by ribs formed onto, or sheets that are additionally applied to the substrate. The substrate itself may be locally folded or structured to locally improve the stiffness. The adhesive substrate may be a multilayered substrate including at least one supportive layer and at least two adhesive layers for attaching the housing to a part of the second surface and for attaching the removable release liner to the first surface.

The supportive layer may be a woven or non-woven (for example fleece type) material, constructed of an organic material such as cotton. The supportive layer may be constructed from a flexible material. The open spaces in the woven material may improve penetration and adhesion of the adhesive layers and may be beneficial for the mechanical flexibility. In another embodiment a plurality of supportive layers may be used having internal adhesive layers between supportive layers and external adhesive layers for attachment to the release liner and housing, respectively. The skin adhesive layer may be based on an acrylic adhesive which may be biocompatible. The internal layers may be electrically conductive or may have electrical contacts. For example conductive wires may be woven in the layer or conductive ink patterns (for example carbon or silver) may be printed onto the layers. Electrically conductive layers may be used as sensors within the substrate such as a capacitive sensor or proximity sensor or a temperature sensor.

The removable release liner may be constructed from a sheet of an organic material such as paper, coated paper or from a polymeric material for example polyethylene terephthalate (PET) or an olefinic polymer (Polypropylene or Polyethylene). The multilayered adhesive substrate and/or the release liner may have one or more apertures, for example allowing movement of an insertion needle from inside the housing to outside the housing through the aperture.

The substrate may have another cut-out, defined by the fact that the release liner does not extend therefrom for easy liner removal but, instead, the release liner follows the contour lines of this another cut-out. Such another cut-out has a different purpose and is not related to the ease of liner removal but may facilitate device assembly.

Therefore a plurality of cut-outs may be envisaged, which either facilitate liner removal and/or facilitate assembly of the device.

The housing may be an outside or exterior housing including a top housing part and a bottom housing part and the adhesive substrate may be attached to the bottom housing part.

The second surface of the substrate may be attached to the bottom housing thereby forming the second surface area of the substrate. The bottom and top housing may be permanently attached to each other or the top housing is releasably connected to the bottom housing. The latter option may be beneficial for a semi-disposable device having a re-usable part (connected to or integrated with the top housing) and a disposable bottom housing connected to the substrate with the release liner. Part of the device may be disposed after use and a part of the device may be re-used for another medicament injection or infusion. The corners of the housing are at least located in the bottom housing part.

The patch injection device may have features to facilitate the removal of the device from the skin after skin attachment. As the flexible skirt surrounding the housing completely attaches to the skin, it may be difficult to remove the device by the user.

The patch injection device may include a non-adhesive area in the substrate to promote removal of the patch injection device after skin attachment. The non-adhesive area is provided in the first surface of the substrate. The non-adhesive area may be surrounded by adhesive for skin attachment and there may be a gradual change from adhesive area to the non-adhesive area.

The non-adhesive area may be located in the skirt of the substrate forming a winglet such that the winglet is not attached to the skin and may be grasped by the user for removal of the patch injection device from the skin. The non-adhesive area or winglet may be located adjacent from the pull tab of the release liner when the release liner is attached to the first surface of the substrate. The winglet may be located at a corner of the patch injection device.

The surface area of the non-adhesive area may be less than 50% of the first surface area, or less than 20% or less than 10% of the first surface area. In an embodiment the non-adhesive area amounts to 5% of the first surface area.

The surface opposite to the non-adhesive area or the top surface of the winglet that is within the skirt may include an indicator. The indicator may be textured for a haptic touch and/or may have a visual indicator. The visual indicator may be, an arrow indicating the direction for removal of the patch injection device. Additionally or alternatively, the indicator may have include a text message. Thus there may be an indicator on the release liner facilitating liner removal before injection and there may be an indicator on the winglet for removal of the device form the skin after an injection. The two indicators may be positioned adjacent to each other.

The housing of the patch injection device may enclose a drive mechanism for expelling a medicament, a control unit for controlling medicament delivery, and a fluid path including an injection needle for delivery of the medicament.

The drive mechanism may include an electric motor, or the mechanism may use a different power package such as a spring, or the mechanism is hydraulic driven or driven by (gas) pressure. The drive mechanism may include a piston rod for advancing a plug in a reservoir towards an outlet of the reservoir. A gear mechanism may be positioned between the electric motor and the piston rod converting rotational movement of the electric motor in linear movement of the piston rod. The piston rod may be a segmented piston rod and each segment may be connected by a film link to another segment such that the piston rod can reversibly change from a linear to a curved configuration. The control unit may be constructed as a mechanical system, for example using motion links, or the control unit may be an electronic control unit located on a printed circuit board and including integrated circuits and a memory unit or a transmitter/receiver unit. The patch injection device includes a battery when an electric motor and an electric control circuit is used.

The fluid path may include a flexible tubing fluidly connecting two hollow insertion needles, one insertion needle for insertion into the skin of the patient and the other needle for insertion into the reservoir to establish a fluid connection between the reservoir and the skin insertion needle. The reservoir needle of the fluid path may be fixed in the housing and the reservoir may be moved within the housing to establish the fluid connection or the needle may be moved with respect to a reservoir that is fixed to the housing. This so-called reservoir needle is configured to be moved by a reservoir needle insertion mechanism. The skin needle may be fixed to and extend from the housing and may be covered by a moveable needle cover sleeve which is moved with respect to the housing for skin insertion. Alternatively, the skin needle may be arranged within the housing to be moved from a retracted position into an extended position outside the housing. The movement may be reversible or a one-way movement. A skin needle insertion mechanism inserts the skin needle and the needle insertion mechanism may be biased by a spring. The skin needle insertion mechanism may also provide the force for the needle retraction after the medicament has been delivered.

Furthermore, the housing of the patch injection device may house a cartridge including a fluid medicament. The cartridge may include a flexible reservoir or a rigid reservoir. Examples of a rigid reservoir may be a glass or polymer cartridge (for example constructed from cycloolefinic copolymer (COC). The flexible or rigid reservoir may be closed by a pierceable septum configured to be pierced by the reservoir needle. Alternatively, the reservoir may be permanently connected to the fluid path or to the reservoir. A rigid cartridge may be closed on one end by a plug or plunger such that the fluid medicament is enclosed between the plug and the septum. The cartridge may be present in the device which may facilitate a prefilled and ready to use device that may restricted to single use only. Alternatively, the cartridge may be inserted into the device just prior to use. The cartridge may be inserted via a door mechanism including a cartridge holder or cover adapted to receive the cartridge. The door mechanism may include a cartridge needle for penetrating the septum when the cartridge is inserted into the door mechanism. As a further option, the door mechanism may include a latching mechanism which, once latched, mechanically or electrically unlocks the device from a dormant state into an active state such that the delivery device is ready to use after cartridge insertion. Insertion of the cartridge just prior to use may facilitate the use of delicate medicaments that need to be dissolved, mixed or reconstituted outside the device just prior to use.

A patch injection device that is of the semi-disposable type may include a disposable part having the bottom housing part with the adhesive substrate, the release liner and the components that may contact the fluid medicament such as the cartridge and the fluid path. The cartridge is prefilled and present within the disposable part, or the cartridge is prefilled but inserted into the disposable part before the disposable part is adhered to the skin, or the cartridge is within the disposable part and empty (not prefilled) and the user has to fill the cartridge in the disposable part of the device prior to use. The disposable part may include a battery source to power the drive mechanism in the reusable part.

The reusable part may include the drive mechanism, the control unit, and the electric motor. The disposable and reusable part may be releasable mechanically connected or connectable to each other via a snap fit connection, or via a bayonet connection or via a screw type of connection. The disposable and reusable part may be connected by a third component, a clip or assembly member. Next to the mechanical connection, there may be an electrical connection between the reusable and disposable parts and the electrical connection may be separate from, or integrated with, the mechanical connection.

### BRIEF DESCRIPTION OF THE DRAWINGS

FIG. 1 depicts a bottom view for a prior art device according to WO17219154.

FIG. 2 depicts a top view of a patch injection device according to the present disclosure including a housing and an adhesive substrate with release liner.

FIG. 3 depicts a top view of a patch injection device including a housing and an adhesive substrate; The release liner has been removed from the adhesive substrate.

FIG. 4 depicts a cross section of the housing, the substrate and the release liner.

FIG. 5 presents detailed area “C” of FIG. 4 depicting a corner of the housing, the substrate, the cut-out and the release liner.

FIG. 6 depicts a top view of the substrate and release liner.

FIG. 7 depicts a detail for the layered substrate in a cross sectional view.

FIG. 8 demonstrates the removal of the release liner using one hand for the liner and the other hand for holding the housing.

FIG. 9 depicts a winglet on the patch injection device facilitating removal of the patch injection device from the skin.

### DETAILED DESCRIPTION

### Definitions

The term “medicament” or “medication” includes any flowable medical formulation suitable for controlled administration through a means such as, for example, a cannula or a hollow needle and includes a liquid, a solution, a gel or a fine suspension containing one or more medical active ingredients. A medicament can be a composition including a single active ingredient or a pre-mixed or co-formulated composition with more than one active ingredient present in a single container. Medication includes drugs such as peptides (for example insulin, insulin-containing drugs, GLP-1 containing drugs or derived or analogous preparations), proteins and hormones, active ingredients derived from -or harvested by- biological sources, active ingredients based on hormones or genes, nutritional formulations, enzymes and other substances in both solid (suspended) or liquid form but also polysaccharides, vaccines, DNA, RNA, oligonucleotides, antibodies or parts of antibodies but also appropriate basic, auxiliary and carrier substances.

The distal end or distal direction is defined by the direction of the needle configured to penetrate the skin of the patient. For a patch injection device the distal end and the distal direction is towards the needle configured to penetrate the skin of the patient, which may be along the axis of the device or tilted or perpendicular to the axis of the device. The proximal direction or end is opposite to the distal direction or end.

A patch injection device 1 according to the prior art (WO2017219154) is depicted in FIG. 1. The patch injection device 1 includes a housing 2 which may be attached to the skin of a patient using an adhesive substrate 5. Prior to use, the adhesive substrate 5 may be covered by a removable release liner 10 which extends laterally in one direction beyond the dimensions of the housing 2 forming a pull tab 12 including an opening or hole 14. The user can hold the device in one hand and grip the pull tab 12 with his other hand using the hole 14 as a pull ring to remove the release liner 10 in the direction indicated by the arrow. The user does not have to peel the release liner 10 directly from the substrate 5 as the adhesive substrate does not extend beyond the housing and can hold the device while removing the release liner and this may facilitate simplified device handling. For large dose patch injectors, the available surface area for skin attachment is, for this example, limited to the adhesive substrate 5 covering the bottom surface of a bottom housing part 4 and the weight of the device may call for stronger adhesives for skin attachment to prevent premature detachment of the device.

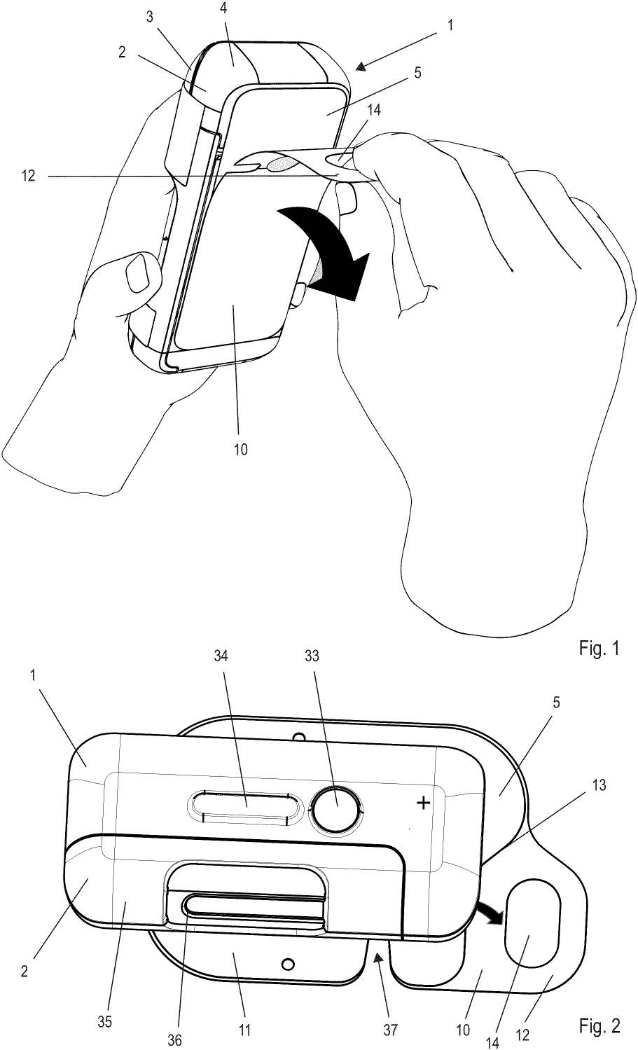

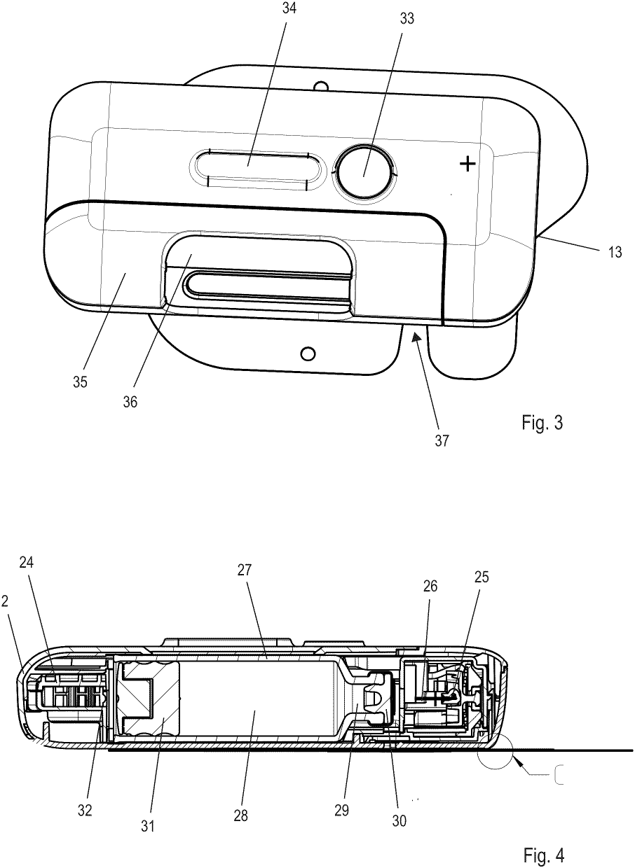

A patch injector 1 according to the present disclosure is presented in FIGS. 2 to 8. The adhesive substrate 5 may at least partially surround the housing 2 thereby establishing a skirt 11 thereby increasing the surface area available for skin attachment. The adhesive substrate has a first surface contacting the release liner 10 and a second surface contacting the housing 2, or may contact the bottom housing 4. The first surface facing the liner may be fully covered with a skin adhesive whereas the second surface facing the housing has no adhesive in the free standing area of the skirt. The skirt 11 includes a cut-out 13. Drawing the virtual contour for the substrate 5 would result in a rectangle with four rounded corners and the cut-out 13 is defined by an area of the substrate 5 that has been left out in the one corner. The removable release liner 10 may be attached to the substrate as depicted in FIG. 2 or has been removed as depicted in FIG. 3. The removable release liner 10 includes a pull tab 12 which extends from the housing 2 and the adhesive substrate. The pull tab 12 may have an opening or hole 14 such that the pull tab can be easily gripped by the user. In the embodiment presented in FIG. 2, the pull tab 12 is unitarily formed with the removable release liner 10. The pull tab 12 may also be available as a separate part that is attached or connected to the release liner 10. The release liner may include an arrow or sign indication the direction for removing the release liner. Such an arrow or indicator may be printed or embossed on both sides of the release liner in the area of the cut-out 13 of the substrate. Alternatively, the opening or hole itself 14 may have a different shape, for example an “arrow shape” indicating the direction for liner removal. The indicator on the release liner may start at the location where the cut-out 13 of the substrate is adjacent or proximate to the housing 2 or bottom housing 4.

The substrate 5 and the release liner 10 may have a second cut-out 37 in skirt 11. The release liner 10 follows the contours of the substrate 5 and no pull tab is formed. The second cut-out may facilitate improved assembly of the device, for example for sideways insertion of sterile barriers or isolation foils for battery terminals or for providing access to sterilization agents such as ETO. The second cut-out 37 is therefore referred to as an assembly cut-out and may be independent from the above mentioned cut-out 13 promoting release liner removal prior to use.

There may be a plurality of cut-outs 13 available for promoting release liner removal.

The patch injection device 1 in FIG. 2 may further include an actuation button 33 and/or an optical indicator 34 (for example a LED indicator) and/or a cover 35 for closing a cartridge holder or covering a cartridge present within the housing. The cover 35 may be a removable cover and may have a viewing window 36 for viewing the cartridge present in the patch injection device.

A cross section of the patch injection device is depicted in FIG. 4 with the cartridge 27 containing a fluid medicament 28 present between a moveable piston 31 and a pierceable septum 30. The medicament may be a solid medicament, for example a lyophilisate that may be reconstituted prior to use. A drive mechanism 24 including drive train and a piston rod 32 is configured to advance the piston 31 in the cartridge towards an outlet 29 which is closed by the septum 30. The housing 2 furthermore houses a fluid path 25 including at least one hollow injection needle 26. The at least one injection needle 26 may be connected to a tubing for fluid delivery from the cartridge towards an outlet of the fluid path. The fluid path 25 may include two hollow injection needles, one for piercing the septum 30 and the other one for piercing the skin of the patient. One needle may be aligned with the longitudinal axis of the cartridge 27 and the other needle may be oriented perpendicular thereto. A needle insertion mechanism may be configured to move the needle 26 from a retracted position (depicted in FIG. 4) to an inserted position piercing the septum. The needle insertion mechanism may simultaneously move a tip of the second needle from a retracted position inside the housing through an aperture 40 in the housing and substrate to a position exterior to or outside the housing for penetrating the skin of a patient.

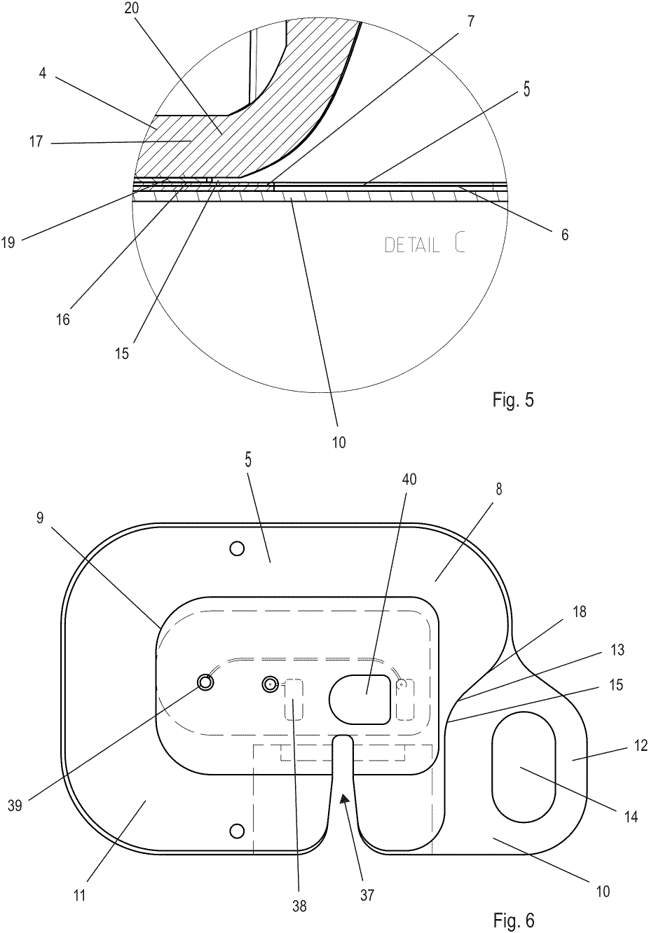

A detailed view for the cross section of FIG. 4 is depicted in FIG. 5 presenting the interface between a corner 20 of the bottom housing 4, the adhesive substrate 5 and the release liner 10. The cross section 17 of the housing includes a substantially flat bottom surface being part of the contacting surface 19 between the housing and the substrate. Due to the presence of the cut-out 13, the substrate 5 is also depicted having a local cross section 16 (hatched) whereas the part of the substrate 5 that is out of the cross section plane is indicated as non-hatched. The cut-out 13 reaches or is adjacent to the housing indicated as location 15. The indication arrows on the release liner 10 may start at location 15.

A top view of the substrate 5 and the release liner 10 is depicted in FIG. 6, showing the first surface area 8 of the substrate 5 contacting the release liner 10 and the second surface area 9 for contacting the housing. The excess area or skirt 11 is configured to surround the housing having the cut-out 13 and a second cut-out 37. The cut-out 13 is formed as an arc 18 and the arc 18 comes close to or is adjacent to the second surface area 9 intended for fixation of the housing to the substrate, presented in FIG. 6 as the location 15. The pull tab 12 extends from the cut-out 13. The substrate may further include at least one sensor area 38 and/or at least one electrical contact or wiring 39. The sensor may be a proximity or contact sensor which may be a mechanical sensor or an electrical sensor, for example a capacitive sensor. Furthermore, the substrate 5 may have the aperture 40 for the skin insertion needle.

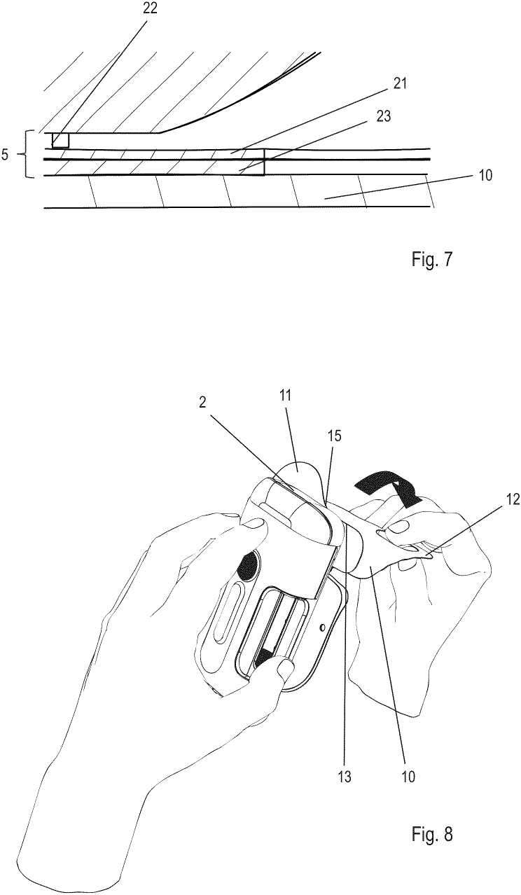

A detail of the layered substrate 5 is depicted in FIG. 7. The layered substrate 5 includes at least one adhesive layer 22 for contacting the housing and at least one supportive layer 21 and at least one skin adhesive layer 23. The supportive layer 21 may be positioned between the skin adhesive layer 23 and the adhesive layer for contacting the housing 22.The substrate may constructed from a plurality of double sided adhesive tapes, each having a supportive layer and two adhesive layers adapted to the specific purpose, e.g. either an internal adhesive layer for mechanical support or an external adhesive layer for contacting the housing or the release liner.

The skirt 11 may be locally mechanically supported by the substrate itself (for example by a rippled, or folded substrate structure) or the skirt may be locally stiffened by adding ribs or films onto the second surface facing the housing.

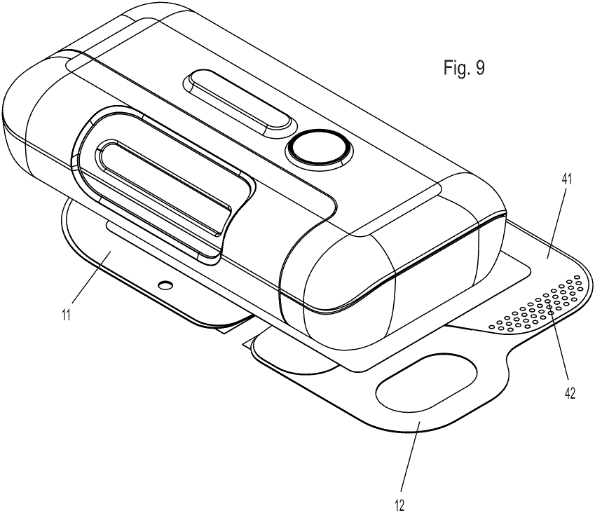

The removal of the release liner is depicted in FIG. 8. The user holds the housing 2 of the patch injection device 1 in one hand and may grip the pull tab 12 with his other hand. The patch injection device includes the skirt 11 of the substrate 5 surrounding the housing and the pull tab 12 is formed as an extension of the release liner 10. The cut-out 13 of the substrate 5 together with the pull tab 12 ensures that the pull tab can be gripped on both sides by the user. By pulling the pull tab in the direction indicated by the arrow, the mechanical load is directed to location 15 were the cut-out is adjacent to the housing. The housing or the bottom housing part may provide mechanical support to the flexible substrate 5 as the substrate 5 is attached to the bottom housing. The peel-off force releasing the liner from the substrate may be directed to the housing via the substrate at the location were the cut-out is adjacent to the housing. The release liner may first start to peel from the substrate where the substrate is mechanically supported and avoiding the skirt area which is not mechanically supported by the housing. The intended starting point for the liner removal may be location 15 and the liner removal will subsequently progress to the other areas, mechanically supported by the housing and finally to the non-mechanically supported areas in the flexible skirt. With this configuration, an easy removal of the release liner may be envisaged while holding the housing containing the medicament.

The patch injection device may have a feature to facilitate the removal of the device from the skin after skin attachment as depicted in FIG. 9. The adhesive substrate 5 may include a winglet 41 that may be part of the skirt 11 and the area on the winglet facing the release liner before liner removal is not covered by a skin adhesive whereas the remaining part on the first surface of the substrate is covered with a skin adhesive. The winglet 41 may be located next to the pull-tab 12. After liner removal, the first surface of the substrate attaches to the skin except for the winglet. The winglet may be easily grasped by the user for removal of the device from the skin once the medication has been injected. The top surface of the winglet facing the user may comprise an indicator, for example dots 42.The dots may be embossed into the substrate or printed onto the substrate to increase the roughness such that the load can be easily transferred from the user’s fingers to the substrate.

In the claims, the word “including” does not exclude other elements or steps, and the indefinite article “a” or “an” does not exclude a plurality. For example “substrate”, “liner” or “adhesive” does not exclude the fact that there may be two “liners”, “substrates” or “adhesives” that functionally or structurally fulfill the purpose of “a liner” or “a substrate” or “an adhesive”.

LIST OF REFERENCE SIGNS

| | | | |
|---|---|---|---|
| 1 | Patch injection device | 20 | Corner housing |
| 2 | Housing | 21 | Supportive layer |
| 3 | Top housing | 22 | Adhesive layer to housing |
| 4 | Bottom housing | 23 | Skin adhesive |
| 5 | Adhesive substrate | 24 | Drive mechanism |
| 6 | First surface | 25 | Fluid path |
| 7 | Second surface | 26 | Injection needle |
| 8 | First surface area | 27 | Cartridge |
| 9 | Second surface area | 28 | Fluid medicament |
| 10 | Removable release liner | 29 | Outlet |
| 11 | Skirt | 30 | Septum |
| 12 | Pull tab | 31 | Piston |
| 13 | Cut-out | 32 | Piston rod |
| 14 | Opening or Hole | 33 | Actuation button |
| 15 | Location where cut-out reaches housing | 34 | Optical indicator |
| 16 | Cross section substrate | 35 | Cover |
| 17 | Cross section housing | 36 | Viewing window |
| 18 | Arc | 37 | Second cut-out |
| 19 | Contacting surface housing- substrate | 38 | Sensor area |
| 39 | Electrical contacts / wiring | | |
| 40 | Aperture | | |
| 41 | Winglet | | |
| 42 | Dots | | |

What is claimed is:

1. A patch injection device, comprising:
a housing;
an adhesive substrate having a first surface with a first surface area for skin attachment and a second surface which is opposite the first surface for attaching partly to the housing; and
a removable release liner,
wherein a surface area of the second surface attaching to the housing is smaller than the first surface area and wherein an excess area defines a skirt of the substrate and at least partially surrounds the housing, and
wherein the first surface of the substrate attaches to the removable release liner which fully covers the first surface area of the substrate,
characterized by a pull tab that is part of the release liner and which is located in a cut-out of the skirt of the substrate, such that a peel force is transmittable from the release liner to the housing at a location where the cut-out reaches the housing.

2. The patch injection device according to claim 1, wherein the release liner partially extends beyond the adhesive substrate in an area of the cut-out, such that a surface area of the release liner is larger than the first surface area.

3. The patch injection device according to claim 2, wherein the adhesive substrate is flexible.

4. The patch injection device according to claim 3, wherein the pull tab is integrally formed with the release liner or the pull tab is a separate part attached to the release liner.

5. The patch injection device according to claim 4, wherein the pull tab comprises an opening.

6. The patch injection device according to claim 1, wherein the peel force is transmittable to the housing via the adhesive substrate at the location where the cut-out reaches the housing.

7. The patch injection device according to claim 6, wherein the cut-out is arc shaped.

8. The patch injection device according to claim 7, wherein the arc shaped cut-out of the skirt is adjacent or congruent to a contacting surface between the housing and the substrate.

9. The patch injection device according to claim 8, wherein the housing comprises at least one corner and the at least one corner is adjacent to the arc shaped cut-out of the skirt.

10. The patch injection device according to claim 9, wherein the adhesive substrate is partially stiffened in the skirt surrounding the housing.

11. The patch injection device according to claim 10, wherein the release liner is split into two separate parts each having a separate pull tab.

12. The patch injection device according to claim 11, wherein the adhesive substrate is a multilayered substrate comprising at least one supportive layer and at least two adhesive layers for attaching the housing to a part of the second surface and for attaching the removable release liner to the first surface.

13. The patch injection device according to claim 12, wherein the housing is an exterior housing comprising a top housing part and a bottom housing part, and wherein the adhesive substrate is attached to the bottom housing part.

14. The patch injection device according to claim 13, wherein the housing encloses a drive mechanism for expelling a medicament, a control unit for controlling medicament delivery and a fluid path comprising an injection needle for delivery of the medicament.

15. The patch injection device according to claim 14, wherein the housing houses a cartridge comprising a fluid medicament.

16. A patch injection device, comprising:
a housing;
an adhesive substrate having a first surface with a first surface area for skin attachment and a second surface, which is opposite the first surface for attaching partly to the housing; and
a removable release liner,
wherein a surface area of the second surface attaching to the housing is smaller than the first surface area and wherein an excess area defines a skirt of the substrate and at least partially surrounds the housing, and
wherein the first surface of the substrate attaches to the removable release liner which fully covers the first surface area of the substrate,
characterized by a pull tab that is a part of the release liner and which is located in a cut-out of the skirt of the substrate such that a peel force is transmittable from the release liner to the housing at a location where the cut-out reaches the housing,
wherein the peel force is transmittable to the housing via the adhesive substrate at the location where the cut-out reaches the housing.

17. The patch injection device according to claim 16, wherein the cut-out of the skirt is arc shaped and the arc shaped cut-out is adjacent or congruent to a contacting surface between the housing and the substrate.

18. The patch injection device according to claim 17, wherein the adhesive substrate comprises a winglet that is not covered by the skin adhesive, whereas a remaining part on the first surface of the substrate is covered with the skin adhesive.

19. The patch injection device according to claim 18, wherein the housing comprises at least one corner and the at least one corner is adjacent to the arc shaped cut-out of the skirt.

20. The patch injection device according to claim 19, wherein the pull tab comprises an opening and the pull tab is integrally formed with the release liner or the pull tab is a separate part attached to the release liner.
